(19) United States
(12) Patent Application Publication
(10) Pub. No.: US 2022/0092785 A1
(43) Pub. Date: Mar. 24, 2022

(54) METHOD OF DECOMPOSING A RADIOGRAPHIC IMAGE INTO SUB-IMAGES OF DIFFERENT TYPES

(71) Applicant: AGFA NV, Mortsel (BE)

(72) Inventors: Jeroen Cant, Mortsel (BE); Joris Soons, Mortsel (BE)

(21) Appl. No.: 17/414,439

(22) PCT Filed: Dec. 16, 2019
(86) PCT No.: PCT/EP2019/085328

§ 371 Date: Jun. 16, 2021

(30) Foreign Application Priority Data
Dec. 18, 2018 (EP) 18213257.1

(51) Int. Cl. G06T 7/11; G06V 10/764; G06V 10/82; A61B 6/00

(52) U.S. Cl. CPC G06T 7/11; G06V 10/765; G06V 10/82; A61B 6/52; G06T 2207/10116; G06V 2201/03; G06T 2207/20084; G06T 2207/20212; G06T 2207/20081; G06T 2207/20021

(57) ABSTRACT

Digital signal representations of sub-images are obtained by applying an optimization process wherein a sum is minimized, the sum having a first term representing a measure of the consistency of the sum of a digital representations of sub-images with said radiographic image and wherein the second term is a sum of cost functions each describing the type of one of said sub-images.

### FIELD OF THE INVENTION

The present invention is in the field of digital radiography. More in particular the invention relates to a method of decomposing a digital representation of a radiographic image into sub-images of different types which may be differently processed or differently classified.

### BACKGROUND OF THE INVENTION

Due to their projective nature, X-ray images are difficult to analyze.

Contrary to regular photographic images, image pixels in transmission images (e.g. X-ray images) contain information about all the different structures that were encountered by X-rays when passing through the patient onto an image detector. Examples of such structures and different materials which are encountered in the case of a radiation image of a human are bone, soft tissue, air, metallic implants, collimators to block part of the radiation, etc.

As these structures are projected on top of each other in an X-ray projection image, a straightforward edge detection is often not sufficient to segment the different parts of the imaged patient or object.

This superposition of structures also poses additional difficulties for image processing (e.g. for histogram analysis), compared to regular photographic or video images which usually contain opaque objects.

It is an aspect of the present invention to provide an enhanced method of decomposing a radiographic image into sub-images of different types.

### SUMMARY OF THE INVENTION

The above-mentioned aspects are realized by a method having the specific steps set out in claim 1.

Further embodiments of the invention are set out in the dependent claims.

Advantages and further embodiments of the present invention will become apparent from the following description and drawings.

In this invention, the projected image Im is regarded as a sum of different sub-images Imᵢ of different types.

In the context of this invention with notion ‘types’ refers to different items that are superposed in the projected image because they are encountered successively by a beam of radiation which is used to generate the radiographic image.

Examples are a collimator collimating the radiation emitted by a source of x-rays, bone, soft tissue, implant images . . . .

Also effects generated by the characteristics of the x-ray imaging process such as radiation scattering, noise, Heel effect, implant image . . . are considered types of sub-images.

Consequently an image can be described as a sum of such sub-images.

For example:

Im=Im_{collimator}+Im_{bone}+Im_{soft tissue}+Imᵢₘₚₗₐₙₜₛ+Imₙₒᵢₛₑ+Im_{scatter}+Im_{heel effect}+Im . . .

The representation of Im as a sum of sub-images Imᵢ can be justified intuitively, as the attenuation of an X-ray beam when traversing different materials is described by the law of Beer Lambert:

I=I₀e^{-∫μ(x)dx} (1)

with I the unattenuated X-ray intensity, measured at the detector, I° the measured X-ray intensity at the detector after traversing different materials with attenuation coefficient μ, and x a position along the x-ray beam.

After a log transform, eq (1) can be written as

$$- log ( \frac{I}{{I}_{0}} ) = \sum μΔ x$$

where the log transformed and intensity corrected image represents the sum of the different attenuation values of the encountered tissues.

The goal of decomposing the image Im into different image components Imᵢ is to design a more efficient image processing P for Im, i.e. processing can be adapted to each of the sub-images.

An example of such an image processing P is to reduce the weight of Im_noise, Im_scatter, Im_Heel effect and thus obtain a noise reduced version of Im.

In another example, a specific contrast improvement could be applied to Im_bone, which does not affect (i.e. introduce artifacts in) Im_soft tissue.

In still another example analysis can be applied on the sub-images to steer image processing.

In general, a content specific processing Pᵢ could be applied to the different sub images Imᵢ, resulting in an optimal processing P of the image Im:

$$P ( Im ) = {\sum }_{i} {P}_{i} ( {Im}_{i} )$$

A second potential benefit of decomposing the image Im into different sub-image Imᵢ, is to facilitate a detection, segmentation or classification task D.

Automatic detection tasks Dᵢ might perform more optimally on the different sub-image Imᵢ, without being hindered by non relevant content of the other sub images.

As an example, an automatic detection of soft tissue abnormalities could benefit from the absence of bone or implants in the image.

The method of the present invention is generally implemented in the form of a computer program product adapted to carry out the method steps of the present invention when run on a computer. The computer program product is commonly stored in a computer readable carrier medium such as a DVD a hard disk or the like. Alternatively the computer program product takes the form of an electric signal and can be communicated to a user through electronic communication.

Further advantages and embodiments of the present invention will become apparent from the following description.

### DETAILED DESCRIPTION OF THE INVENTION

In this invention, an image Im is decomposed into different sub images Imᵢ such that

$$\vert Im - {\sum }_{i} {Im}_{i} \vert < ϵ \qquad ( 2 )$$

with ε is a constant to allow a fault tolerance, and 0<i<N, with N the number of sub images Imᵢ.

The constraint in Eq. 2 could also be written as

$$\vert Im - {\sum }_{i} {Im}_{i} \vert = 0$$

in which case no faults are tolerated.

For each sub image Imᵢ, a specialized image processing task Pᵢ or classification task Dᵢ could be designed, which might perform better than their counterparts P and D working on the original image Im.

The inverse problem as defined in Eq. (2) is highly underdetermined.

An infinite number of correct but random images Imᵢ can be generated, of which the sum results in Im.

To guarantee that each sub image Imᵢ corresponds to a target sub class of images (e.g. bone images), a cost function Lᵢ is created which expresses prior knowledge for a given sub image (e.g. characteristics of a typical bone image)

An example of Lᵢ could be a smoothness constraint, a Total Variation constraint, a similarity metric with a prior image, etc.

The inverse problem can thus be written as:

$$\vert Im - {\sum }_{i} {Im}_{i} \vert + {\sum }_{i} {β}_{i} {L}_{i} ( {Im}_{i} ) < ϵ \qquad ( 3 )$$

where the first term measures the consistency with the original image Im, and the second term sums up the cost functions Lᵢ of the different sub images Imᵢ, with a weight pᵢ.

### Design of Cost Functions Lᵢ.

The cost functions Lᵢ describe how well the sub image Imᵢ, fits into the desired category i.

It is of critical importance that the cost functions Lᵢ efficiently describe the desired category, as otherwise the decomposition of Im will result in meaningless sub images Imᵢ.

For example, if Imᵢ should represent the collimator, the corresponding Lᵢ could enforce a piecewise constant image, consisting of only 2 intensities (corresponding to metal and air).

A possible cost function to express that the values of Im_{L} should belong to a discrete set of J values aⱼ, with j∈[1 . . . J], is

Lᵢ(Imᵢ)=Σ_{x,y} minⱼ|Im_{x,y}−aⱼ|, where

aⱼ represents a value in the image that is to be expected based on prior knowledge.

As an example, in the case of if Imᵢ representing the collimator, a₀ could be 0 and a₁ could be set equal to a predefined value. A possible method to derive a₁ could be to acquire a representative flat field exposure, containing the collimator shape. After log transform of the image, a₁ could e.g. be derived as the difference between the average pixel values in the non-collimated and collimated area of the image.

In another implementation, aⱼ could be derived based on image statistics of Imᵢ itself. E.g. each aⱼ represents one of the most occurring pixel values in Imᵢ. In the case of Imᵢ representing the collimator, aₒ could be set to 0 and a₁ would represent the pixel value with the highest occurrence based on a histogram analysis of Imᵢ.

Another way to express piecewise constancy in a cost function is

Lᵢ(Imᵢ)=Σ_{x,y}|Im_{i,x,y}−Im_{u,x+1,y}|+Im_{i,x,y}−Im_{i,x,y+1}| or,

using the L₂ norm,

$${L}_{i} ( {Im}_{i} ) = {\sum }_{x , y} \sqrt{{( {Im}_{i , x , y} - {Im}_{i , x + 1 , y} )}^{2} + {( {Im}_{i , x , y} - {Im}_{i , x , y + 1} )}^{2}} .$$

Another term, which could be added to most cost functions, is the prior knowledge that all pixel values of Imᵢ should be positive. This can be expressed e.g. as

Lᵢ(Imᵢ)=Σ_{x,y}(|Im_{x,y}|−Im_{x,y})

In general, for any of the desired categories Imᵢ, a cost function Lᵢ could be hand crafted.

Another way to obtain a suitable cost function Lᵢ is through the use of neural networks.

In recent years, much progress has been made in the domain of artificial intelligence. Powerful convolutional networks (CNN) are nowadays capable of classifying images of a vast variety of subjects.

A CNN could be trained to classify images into the different classes of sub images.

The final outcome of this CNN could be a vector of dimension N+1, in which each element represents the match score for sub category i, and the last element the score for not belonging to any of the N categories.

Lᵢ can thus be written in function of the resulting output vector of this CNN:

Lᵢ(Im)=1−CNN(Im)ᵢ

CNN could be trained with relevant examples of the different sub categories. A method to obtain these images is to acquire them experimentally, e.g. acquiring images without any object exposed to obtain a relevant electronic noise image, or acquiring images with only a collimator, or using a phantom which only consists of material from a particular sub class.

Another method to obtain training images for this CNN is to generate projection images virtually, e.g. using CT scans of existing patients/objects.

Existing algorithms for segmentation of tissue types in CT scans could be used to segment the CT scan first. These segmentation algorithms are in general easier to develop, due to the lack of overlap of different structures such as in X-ray projection images.

Subsequently, X-ray projection images Imᵢ of the different sub classes could be simulated from the CT scans, in which only the relevant tissue type i is retained per simulation.

In another embodiment, prior knowledge could be integrated in the cost function using an auto-encoder. A denoising auto-encoder can be trained to represent a subclass of images Imᵢ, e.g. a set of collimation images, bone images, etc. A distance metric could subsequently be calculated between the original Imᵢ and the output of the auto-encoder, assuming that if the image Imᵢ truly belongs to the subclass on which the auto-encoder is trained, the distance will be low. This distance could be used as a cost function Lᵢ,

### Optimization

Once the cost functions Lᵢ are defined, the inverse problem in Eq. (3) can be solved to obtain Imᵢ. Different strategies could be followed to solve this inverse problem.

In a first embodiment, an initial estimate Im_{i,0} is generated. This initial estimate might be a random image, a blank (zero) image, a low pass filtered version of the original image, the result of another image decomposition algorithm (such as a virtual dual energy algorithm, which splits an image Im into a bone and soft tissue image), a trained neural network etc. By choosing β=0, we can keep the initial guess for some sub-images.

Then, the different images Im_{L} are computed iteratively, wherein in each iteration n a new estimate Im_{i,n+1} is computed using the previous estimate Im_{i,n} and a partial derivative image D_{i,n}:

$${D}_{i , n} = \frac{\partial {L}_{i} ( {Im}_{i , n} )}{\partial x \partial y}$$

Im_{i,n+1}=Im_{i,n}+λᵢD_{i,n}
with λᵢ a weight. However, as n progresses, the sum of the sub images

$${\sum }_{i} {Im}_{i , n}$$

will most likely start to diverge from the initial image Im.

Therefore, image consistency operations are needed to ensure the sum of sub images Imᵢ result again in the initial image Im.

This could be achieved in various ways, e.g. by re-distributing the difference over the different components Imᵢ:

Im_{i,n+1}=Im_{i,n}+λᵢDᵢ

Im_{j≠i,n+1}=Im_{j,n}−λᵢ/NDᵢ

Another approach to ensure consistency is to add an additional sub image Im_{N}, which is defined as

$${Im}_{N} = Im - {\sum }_{i = 0}^{N - 1} {Im}_{i} .$$

The optimization problem thus reduces to

$${\sum }_{i = 0}^{N} {β}_{i} {L}_{i} ( {Im}_{i} ) < ϵ$$

in which L_{N} could be a simple norm,
or another measure of the error.

Having described in detail preferred embodiments of the current invention, it will now be apparent to those skilled in the art that numerous modifications can be made therein without departing from the scope of the invention as defined in the appending claims.

1. A method comprising:
decomposing a digital signal representation of an image into a sum of sub-images of different image types selected from the group consisting of a radiographic image, a collimation area image, a bone image, a soft tissue image, a noise image, a scatter image, a heel effect representing image, and an implant image, and
minimizing a first term representing a measure of the consistency of the sum of the sub-images with said image and a second term representing a sum of cost functions of the different sub images, each describing the likeliness of the image being a member of the type of the sub-images, wherein different image processing is applied to said sub-images.

2. The method according to claim 1 wherein said cost functions are weighted by a corresponding weight value.

3. The method according to claim 1 wherein said cost function is obtained through the use of a neural network trained with images of said different types.

4. The method according to claim 1 wherein said cost function is obtained through the use of a neural network trained with phantom images.

5. The method according to claim 1 wherein said cost function is obtained through the use of a neural network trained with simulations of radiographic images.

6. The method according to claim 1 wherein differently processed sub-images are combined to form a combined processed image.

7. The RAM method according to claim 1 wherein a classification task is performed based on one or more of said sub-images.

8. The method according to claim 1 wherein a cost function for a sub-image represents the total variation of the first derivative of the signal representation of the image.

9. The method according to claim 1 wherein said cost function represents a noise measure.

10. The RAM method according to claim 1 wherein said process is initialized with sub-images generated by a trained neural network.

11. A computer program product adapted to carry out the method of claim 1 when run on a computer.

12. A computer readable medium comprising computer executable program code adapted to carry out the steps of claim 1.

13. A computer-readable medium storing processor-executable instructions that, when executed by a processor, configure the processor for:
decomposing a digital signal representation of an image into a sum of sub-images of different image types selected from the group consisting of a radiographic image, a collimation area image, a bone image, a soft tissue image, a noise image, a scatter image, a heel effect representing image, and an implant image, and
minimizing a first term representing a measure of the consistency of the sum of the sub-images with said image and a second term representing a sum of cost functions of the different sub images, each describing the likeliness of the image being a member of the type of the sub-images, wherein different image processing is applied to said sub-images.

14. The computer-readable medium according to claim 13 wherein said cost functions are weighted by a corresponding weight value.

15. The computer-readable medium according to claim 13 wherein the processor-executable instructions comprise instructions for executing a neural network to obtain the cost function, the neural network trained with one or more of images of said different types, phantom images, and simulations of radiographic images.

16. A computer program product comprising processor-executable instructions that, when executed by a processor, configure the processor for:
decomposing a digital signal representation of an image into a sum of sub-images of different image types selected from the group consisting of a radiographic image, a collimation area image, a bone image, a soft tissue image, a noise image, a scatter image, a heel effect representing image, and an implant image, and
minimizing a first term representing a measure of the consistency of the sum of the sub-images with said image and a second term representing a sum of cost functions of the different sub images, each describing the likeliness of the image being a member of the type of the sub-images, wherein different image processing is applied to said sub-images.

17. The computer program product according to claim 16 wherein said cost functions are weighted by a corresponding weight value.

18. The computer program product according to claim 16 wherein the processor-executable instructions comprise instructions for executing a neural network to obtain the cost function, the neural network trained with one or more of images of said different types, phantom images, and simulations of radiographic images.
